(19) United States
(12) Patent Application Publication
(10) Pub. No.: US 2015/0141828 A1
(43) Pub. Date: May 21, 2015

(54) ULTRASONIC DIAGNOSTIC APPARATUS AND ULTRASONIC DIAGNOSTIC METHOD

(71) Applicants: Kabushiki Kaisha Toshiba, Minato-ku (JP); Toshiba Medical Systems Corporation, Otawara-shi (JP)

(72) Inventors: Hiroki Yoshiara, Nasushiobara (JP); Akihiro Kakee, Nasushiobara (JP); Cong Yao, Otawara (JP); Tetsuya Kawagishi, Nasushiobara (JP); Hiroyuki Ohuchi, Otawara (JP); Yasunori Honjo, Otawara (JP); Masahiko Yano, Nasushiobara (JP); Yoshitaka Mine, Nasushiobara (JP)

(73) Assignees: Toshiba Kaisha Toshiba, Minato-ku (JP); Toshiba Medical Systems Corporation, Otawara-shi (JP)

(21) Appl. No.: 14/518,440

(22) Filed: Oct. 20, 2014

(30) Foreign Application Priority Data
Nov. 19, 2013 (JP) 2013-238907

(51) Int. Cl. A61B 8/14; A61B 8/08; A61B 8/00

(52) U.S. Cl. CPC A61B 8/145; A61B 8/54; A61B 8/4444; A61B 8/5207; A61B 8/5253; A61B 8/467; A61B 8/466
USPC 600/447

(57) ABSTRACT

According to one embodiment, an ultrasonic diagnostic apparatus includes a transceiver part, a signal processing part and an image generation part. The transceiver part controls an ultrasonic probe to perform a first and second scans. The first scan transmits a first ultrasonic wave and an ultrasonic wave, obtained by modulating an amplitude of the first ultrasonic wave, to scanning lines distributed three dimensionally, and receives first reflected waves. The second scan transmits a second ultrasonic wave to scanning lines distributed two dimensionally during the first scan, and receives second reflective waves. The transceiver part obtains first and second reception signals based on the first and second reflected waves. The signal processing part generates composite signals by combining the first reception signals. The image generation part generates three dimensional ultrasonic image data based on the composite signals, and two dimensional ultrasonic image data based on the second reception signals.

### CROSS REFERENCES TO RELATED APPLICATIONS

This application is based upon and claims the benefit of priority from Japanese Patent Application No. 2013-238907, filed on Nov. 19, 2013; the entire contents of which are incorporated herein by reference.

### FIELD

Embodiments described herein relate generally to an ultrasonic diagnostic apparatus and an ultrasonic diagnostic method.

### BACKGROUND

Conventionally, as an imaging method using an ultrasonic diagnostic apparatus, a method of performing 3D (three dimensional) contrast volume scan and 2D (two dimensional) B-mode scan alternately is known. A contrast scan is a scan which transmits and receives ultrasonic waves having the multiple rates with injecting an ultrasonic contrast agent, such as microbubbles, and performing the phase modulation or the like, to generate an image using the harmonic signals which is a nonlinear component. The imaging method which creates an image using the harmonic signals with injection of an ultrasonic contrast agent is called CHI (contrast harmonic imaging) or contrast echo method. On the other hand, a B-mode scan is a scan which transmits and receives ultrasonic waves each having a low sound pressure to generate a B-mode image depicting a form, using the signals, which is a linear component, in the fundamental wave band.

When contrast volume scans and B-mode scans are performed alternately, a blood flow is observable in real time by the contrast volume scans, with checking scanning positions with reference to the B-mode images acquired by the B-mode scans.

In the alternate scan which performs contrast scans and B-mode scans alternately, it is desired to make a time phase gap small between the scans performed intermittently in the time phase direction. Especially, when a contrast scan is a 3D volume scan, morphological image data for a monitor are acquired by a B-mode scan after acquisition of contrast volume data by the 3D volume scan. Therefore, a non-negligible time phase gap may arise between contrast volume data acquired by a contrast scan and morphological image data for a monitor acquired by a B-mode scan.

Accordingly, an object of the present invention is to provide an ultrasonic diagnostic apparatus and an ultrasonic diagnostic method, which can acquire 3D contrast volume data of a time series and morphological image data for a monitor with a smaller time phase gap.

### BRIEF DESCRIPTION OF THE DRAWINGS

In the accompanying drawings:

FIG. 1 is a functional block diagram of an ultrasonic diagnostic apparatus according to an embodiment of the present invention;

FIG. 2 is a table showing conditions required according to purposes of acquiring ultrasonic morphological image data;

FIG. 3 is a view showing the first example of a scan sequence for performing a B-mode scan in the middle of a 3D contrast scan;

FIG. 4 is a schematic diagram showing an example of waveforms of transmission signals respectively transmitted in the 3D contrast scan and the B-mode scan shown in FIG. 3;

FIG. 5 is a view showing the second example of a scan sequence for performing a B-mode scan in the middle of a 3D contrast scan;

FIG. 6 is a schematic diagram showing an example of waveforms of transmission signals respectively transmitted in the 3D contrast scan and the B-mode scan shown in FIG. 5;

FIG. 7 is a view showing the third example of a scan sequence for performing a B-mode scan in the middle of a 3D contrast scan;

FIG. 8 is a view showing each update rate of the ultrasonic morphological image data acquired by the B-mode scan and the ultrasonic contrast volume image data acquired by the 3D contrast scan, shown in FIG. 7;

FIG. 9 is a graph showing appropriate frequency characteristics of transmission signals when reception signals acquired by a 3D contrast scan are used only for generation of ultrasonic contrast volume image data;

FIG. 10 is a graph showing appropriate frequency characteristics of transmission signals when a part of reception signals acquired by a 3D contrast scan are used for generation of ultrasonic morphological volume image data;

FIG. 11 is a view describing a method of generating ultrasonic contrast volume image data and ultrasonic morphological volume data by a 3D contrast scan under a three rate AM method;

FIG. 12 is a view describing a method of generating ultrasonic contrast volume image data and ultrasonic morphological volume image data by a 3D contrast scan under a PM method; and

FIG. 13 is a view showing a variation of methods for displaying ultrasonic morphological images for monitor and contrast images.

### DETAILED DESCRIPTION

In general, according to one embodiment, an ultrasonic diagnostic apparatus includes a transceiver part, a signal processing part and an image generation part. The transceiver part is configured to control an ultrasonic probe to perform a first scan and a second scan. The first scan transmits a first ultrasonic wave and an ultrasonic wave, obtained by modulating an amplitude of the first ultrasonic wave, at least one time to each of scanning lines distributed three dimensionally, and receives first reflected waves due to transmissions of the first ultrasonic wave and the ultrasonic wave obtained by modulating the amplitude of the first ultrasonic wave. The second scan transmits a second ultrasonic wave at least one time to each of scanning lines distributed two dimensionally during the first scan, and receives second reflective waves due to transmissions of the second ultrasonic wave. The transceiver part obtains first reception signals based on the first reflected waves and second reception signals based on the second reflected waves, from the ultrasonic probe. The signal processing part is configured to generate composite signals by combining the first reception signals. The image generation part is configured to generate three dimensional ultrasonic image data based on the composite signals, and to generate two dimensional ultrasonic image data based on the second reception signals.

Further, according to another embodiment, an ultrasonic diagnostic apparatus includes a transceiver part, a signal processing part and an image generation part. The transceiver part is configured to control an ultrasonic probe to perform a first scan and a second scan. The first scan transmits a first ultrasonic wave and an ultrasonic wave, obtained by modulating a phase of the first ultrasonic wave, at least one time to each of scanning lines distributed three dimensionally, and receives first reflected waves due to transmissions of the first ultrasonic wave and the ultrasonic wave obtained by modulating the phase of the first ultrasonic wave. The second scan transmits a second ultrasonic wave at least one time to each of scanning lines distributed two dimensionally during the first scan, and receives second reflective waves due to transmissions of the second ultrasonic wave. The transceiver part obtains first reception signals based on the first reflected waves and second reception signals based on the second reflected waves, from the ultrasonic probe. The signal processing part is configured to generate composite signals by combining the first reception signals. The image generation part is configured to generate three dimensional ultrasonic image data based on the composite signals, and to generate two dimensional ultrasonic image data based on the second reception signals.

Further, according to another embodiment, an ultrasonic diagnostic method includes: controlling an ultrasonic probe to perform a first scan and a second scan; obtaining first reception signals based on the first reflected waves and second reception signals based on the second reflected waves, from the ultrasonic probe; generating composite signals by combining the first reception signals; and generating three dimensional ultrasonic image data based on the composite signals and generating two dimensional ultrasonic image data based on the second reception signals. The first scan transmits a first ultrasonic wave and an ultrasonic wave, obtained by modulating an amplitude of the first ultrasonic wave, at least one time to each of scanning lines distributed three dimensionally, and receives first reflected waves due to transmissions of the first ultrasonic wave and the ultrasonic wave obtained by modulating the amplitude of the first ultrasonic wave. The second scan transmits a second ultrasonic wave at least one time to each of scanning lines distributed two dimensionally during the first scan, and receives second reflective waves due to transmissions of the second ultrasonic wave.

Further, according to another embodiment, an ultrasonic diagnostic method includes: controlling an ultrasonic probe to perform a first scan and a second scan; obtaining first reception signals based on the first reflected waves and second reception signals based on the second reflected waves, from the ultrasonic probe; generating composite signals by combining the first reception signals; and generating three dimensional ultrasonic image data based on the composite signals and generating two dimensional ultrasonic image data based on the second reception signals. The first scan transmits a first ultrasonic wave and an ultrasonic wave, obtained by modulating a phase of the first ultrasonic wave, at least one time to each of scanning lines distributed three dimensionally, and receives first reflected waves due to transmissions of the first ultrasonic wave and the ultrasonic wave obtained by modulating the phase of the first ultrasonic wave. The second scan transmits a second ultrasonic wave at least one time to each of scanning lines distributed two dimensionally during the first scan, and receives second reflective waves due to transmissions of the second ultrasonic wave.

An ultrasonic diagnostic apparatus and an ultrasonic diagnostic method according to embodiments of the present invention will be described with reference to the accompanying drawings.

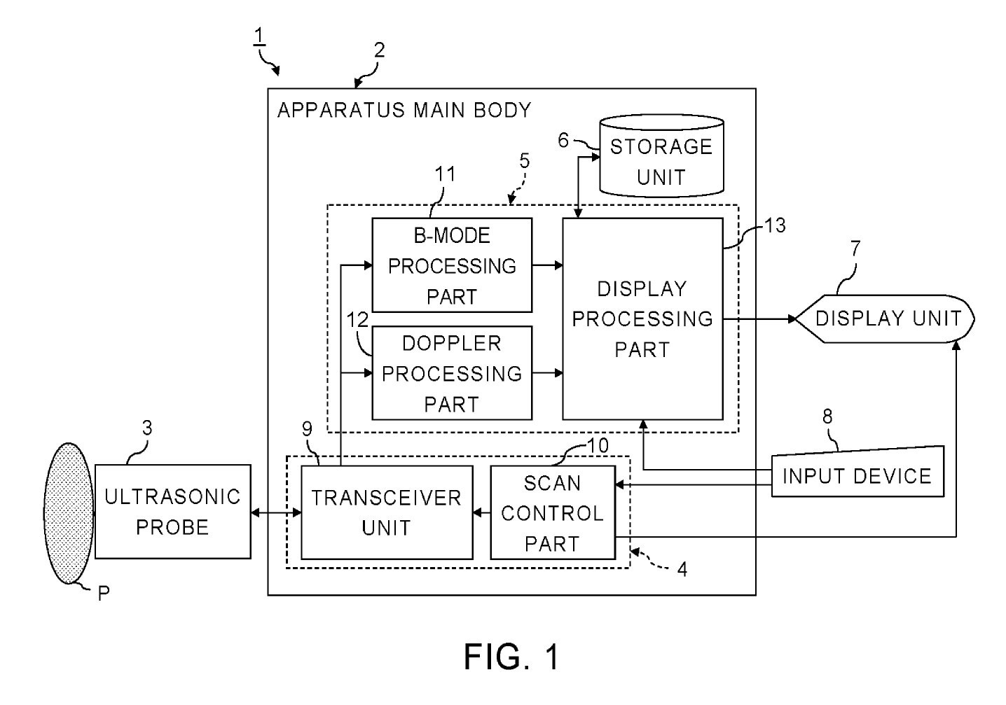

FIG. 1 is a functional block diagram of an ultrasonic diagnostic apparatus according to an embodiment of the present invention.

An ultrasonic diagnostic apparatus 1 is configured by connecting an ultrasonic probe 3 to an apparatus main body 2. The ultrasonic probe 3 has ultrasonic transducers built in, for transmitting and receiving ultrasonic waves toward and from an object P. While each ultrasonic transducer converts a transmission signal, applied as an electric signal, into an ultrasonic signal to transmit the ultrasonic signal inside the object P, each ultrasonic transducer receives an ultrasonic reflected wave which has arisen inside the object P to convert the ultrasonic reflected wave into a reception signal as an electric signal to output the reception signal.

The ultrasonic probe 3 whose ultrasonic transducers are two dimensionally arranged is called a 2D array probe. Meanwhile, the ultrasonic probe 3 whose ultrasonic transducers are arranged in one row and can be swung mechanically is called a mechanical 4D (four dimensional) probe.

The apparatus main body 2 includes a transceiver part 4, a data processing system 5 and a storage unit 6. Further, a display unit 7 and an input device 8 are connected to the apparatus main body 2. The transceiver part 4 has a transceiver unit 9 and a scan control part 10. Meanwhile, the data processing system 5 has a B-mode processing part 11, a Doppler processing part 12 and a display processing part 13.

The transceiver unit 9 has a function to transmit ultrasonic waves by applying a driving pulse, as a transmission signal, to each of the ultrasonic transducers of the ultrasonic probe 3 under control by the scan control part 10, and to generate ultrasonic reception data by receiving a reception signal output from each of the ultrasonic transducers of the ultrasonic probe 3 to perform necessary signal processing of the reception signal under control by the scan control part 10.

A predetermined delay time is given to each of transmission signals applied from the transceiver unit 9 to ultrasonic transducers. Accordingly, an ultrasonic transmission beam which has directivity is formed by ultrasonic signals transmitted from respective ultrasonic transducers. The processing to form an ultrasonic transmission beam by giving a delay time to each transmission signal is also called transmission beam forming. Thus, ultrasonic transmission beams can be transmitted sequentially toward scanning positions by control of the delay times given to the transmission signals.

Similarly, a predetermined delay time is also given to each of the reception signals output from the ultrasonic transducers to the transceiver unit 9. Accordingly, an ultrasonic reception beam which has directivity is formed by ultrasonic reflection echo signals received by respective ultrasonic transducers. The processing to form an ultrasonic reception beam by giving a delay time to each reception signal is also called reception beam forming. Thus, ultrasonic reception beams can be received sequentially from the scanning positions by control of the delay times given to the reception signals.

Then, in the transceiver unit 9, necessary signal processing including A/D (analog to digital) conversion processing and phasing addition processing is performed to each of the reception signals to which delay times have been given. Thereby, ultrasonic reception data corresponding to each of the scanning positions are generated. Moreover, in the transceiver unit 9, reception signals are amplified by an amplifier before delay times are given. Note that, a part of the signal processing performed in the transceiver unit 9 may be performed in the ultrasonic probe 3 side.

When an electronic scanning is performed to form an ultrasonic beam with giving delay times, it is possible to perform a 3D scan which acquires ultrasonic reception data from each scanning position in a 3D region using a 2D array probe. Alternatively, a 3D scan can also be performed by electronic scanning and mechanical swing scanning using a mechanical 4D probe.

Furthermore, when an ultrasonic contrast agent, such as microbubbles, is injected into a blood vessel of the object P and multiple rates of ultrasonic waves which have been modulated by a PM method or the like are transmitted and received, it is possible to perform a 3D contrast scan which receives ultrasonic contrast echo signals reflected by the contrast agent in a 3D scanning region. Moreover, ultrasonic echo signals reflected by a moving matter, such as a blood flow which is moving inside the object P, a contrast agent in the blood flow, or myocardium can be acquired as ultrasonic Doppler signals which have a frequency shift depending on a velocity of a moving matter. Especially, when ultrasonic contrast echo signals are used as ultrasonic Doppler signals, an ultrasonic Doppler image showing a blood flow dynamic state can be generated. The ultrasonic Doppler image on which a blood flow dynamic state is displayed in color is also called color Doppler image.

On the other hand, when ultrasonic reflection signals reflected by structural objects, such as body parts and organs, in the object P are acquired, an ultrasonic morphological image where a form of the structural objects in the object P has been depicted can be generated as a B-mode image.

The scan control part 10 has a function to set a scan sequence which defines transceiver conditions of ultrasonic waves as ultrasonic scan conditions, and a function to perform a scan by controlling the transceiver unit 9 according to the set scan sequence. Especially, the scan control part 10 can control the transceiver unit 9 so that 3D contrast scans for generating ultrasonic Doppler volume image data and B-mode scans for generating B-mode image data are performed alternately using the ultrasonic probe 3.

However, the scan control part 10 is configured to be able to control the transceiver unit 9 under scanning conditions for performing a B-mode scan during a 3D contrast scan. That is, the alternate scan performed under control by the scan control part 10 is a scan which performs a B-mode scan during a 3D contrast scan.

Therefore, ultrasonic signals for ultrasonic Doppler volume image data are received from a part of 3D region which is a target of a 3D contrast scan, and subsequently, ultrasonic signals for B-mode image data are received from a region which is a target of a B-mode scan. After that, ultrasonic signals in all or a part of the remaining part of the 3D region which is the target of the 3D contrast scan are received for the ultrasonic Doppler volume image data.

Therefore, the alternate scan performed under control of the scan control part 10 is a different scan from the conventional alternate scan which starts a B-mode scan after completion of a 3D contrast scan.

Specifically, the 3D contrast scan is the first scan for receiving ultrasonic contrast echo signals, as the first ultrasonic waves for generating ultrasonic contrast volume image data where a contrast agent has been depicted, using the ultrasonic probe 3, from a 3D area of the object P into which the contrast agent has been injected, and subsequently, to obtain the first reception signals as electric signals corresponding to the first ultrasonic waves. Note that, in the 3D contrast scan, ultrasonic waves of which at least one of an amplitude and a phase has been modulated are transmitted, as mentioned above.

Therefore, the 3D contrast scan is the first scan to transmit the first ultrasonic waves and ultrasonic waves that an amplitude of the first ultrasonic waves is modulated by a predetermined ratio, at least one time to each of three dimensionally distributed scanning lines, and subsequently, to receive the first reflected waves based on the transmission, for example. Alternatively, the 3D contrast scan is the first scan to transmit the first ultrasonic waves and ultrasonic waves that a phase of the first ultrasonic waves is modulated by a predetermined ratio, at least one time to each of three dimensionally distributed scanning lines, and subsequently, to receive the first reflected waves based on the transmission, for example. As a matter of course, both an amplitude and a phase may be modulated by predetermined ratios.

On the other hand, the B-mode scan is the second scan to receive ultrasonic reflection echo signals, as the second ultrasonic waves for generating ultrasonic morphological image data where a form of the object P has been depicted, using the ultrasonic probe 3, from the object P, during the 3D contrast scan as the first scan, and to acquire the second reception signals as electric signals corresponding to the second ultrasonic waves. In other words, the B-mode scan is the second scan to transmit the second ultrasonic waves at least one time to each of two dimensionally distributed scanning lines during the first scan, and subsequently, to receive the second reflective waves based on the transmission, for example.

Then, the transceiver part 4, consisting of the transceiver unit 9 and the scan control part 10, is configured to make the ultrasonic probe 3 perform a 3D contrast scan as the first scan and a B-mode scan as the second scan as mentioned above, and to acquire the first reception signals based on the first reflected waves and the second reception signals based on the second reflected waves from the ultrasonic probe 3.

Furthermore, the transceiver part 4 has a function to repeatedly perform a 3D contrast scan as the first scan and a B-mode scan as the second scan using the ultrasonic probe 3 during a predetermined period. Performing such an alternate scan makes it possible to sequentially generate and display time series ultrasonic morphological image data and ultrasonic contrast volume image data in real time.

In addition, the time phase gap between ultrasonic morphological image data and ultrasonic contrast volume image data can be reduced by performing each B-mode scan in the middle of a 3D contrast scan. Specifically, the increase in the time phase gap between ultrasonic morphological image data and the ultrasonic contrast volume image data, which is a problem in the conventional alternate scan that performs every B-mode scan after completion of a 3D contrast scan, is avoidable.

Ultrasonic contrast image data generated as volume data are used for observation of a blood flow dynamic state in a tumor or the like, and also are used for generation of a TIC (Time Intensity Curve). That is, the ultrasonic contrast volume image data become a target of blood flow dynamic state analysis including the generation of a TIC.

In case of generating a TIC of ultrasonic contrast volume image data, setting a VOI (volume of interest) to be a generation target of the TIC is needed. Specifically, a VOI including an observation target, such as a tumor, is set as a generation area of the TIC. Furthermore, when the observation target moves by a beat or breathing, 3D tracking of a VOI including the observation target may be necessary.

However, a portion other than a contrast agent is hardly depicted in ultrasonic contrast volume image data. Therefore, it is unclear which portion of the object P a scanning area is located in, until the contrast agent arrives at the scanning area. Moreover, when a target, such as a tumor, is small, the target may deviate from a scanning area since ultrasonic contrast volume image data are not 2D cross-sectional image data.

Accordingly, an ultrasonic morphological image can be used as a monitor image for performing a position check (orientation) of 3D scanning positions for a 3D contrast scan. Therefore, a B-mode scan as the second scan is performed as a monitor scan.

Moreover, ultrasonic contrast volume image data, in which any portion other than a contrast agent is hardly depicted and a time change in signal values is large, is inappropriate as image data for VOI setting and VOI tracking for generating a TIC of the ultrasonic contrast volume image data. Therefore, in case of performing VOI setting and VOI tracking, referring to ultrasonic morphological volume image data leads to easy VOI setting and easily securing a VOI tracking performance. That is, in case of setting and tracking a VOI, it is preferable to generate ultrasonic morphological volume image data.

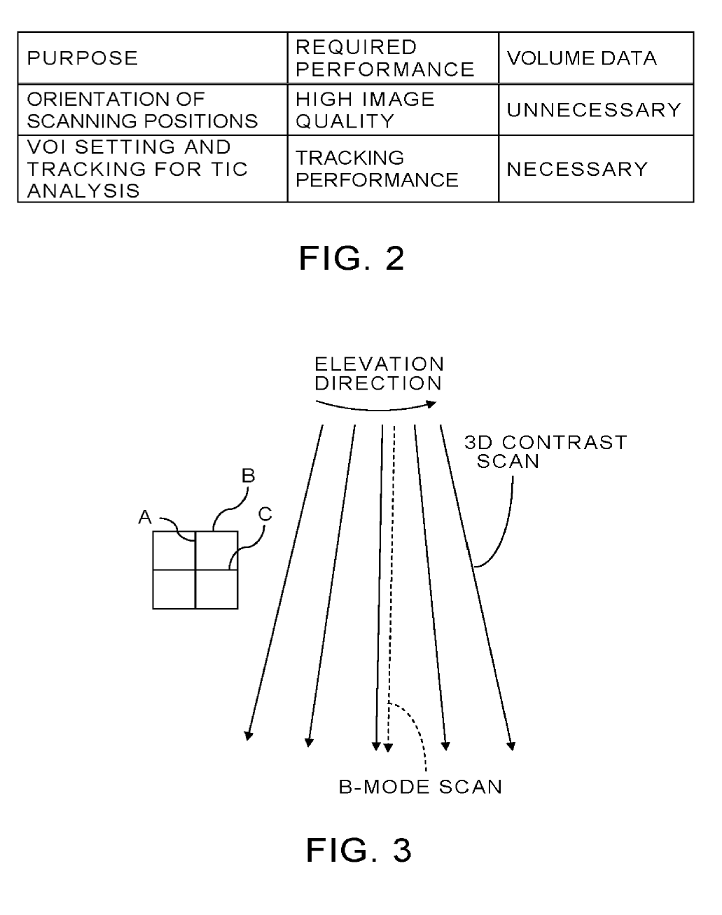

FIG. 2 is a table showing conditions required according to purposes of acquiring ultrasonic morphological image data.

In case of using ultrasonic morphological image data for an orientation of scanning positions in a 4D contrast examination which acquires time series ultrasonic contrast volume image data in real-time, it is necessary to surely keep an imaging target, such as a tumor, in a volume to be a 3D scanning area of the ultrasonic contrast volume image data, with reference to an ultrasonic morphological image displayed as a cross-sectional image of a tissue.

Therefore, displaying a 2D cross-section to be a display target with a high image quality is required for ultrasonic morphological image data for an orientation of scanning positions while volume data is unnecessary as shown in FIG. 2. That is, what is necessary is only to display a 2D tissue cross-section image for an orientation with a high image quality.

On the other hand, in case of using ultrasonic morphological image data for VOI setting and tracking for a TIC analysis of ultrasonic contrast volume image data, it is preferable to acquire the ultrasonic morphological image data as volume data, as mentioned above. However, the image quality of the ultrasonic morphological image data is sufficient so long as a VOI tracking performance can be secured.

However, when the 3D area same as a 3D scanning area by a 3D contrast scan is set as a 3D scanning area by a B-mode scan in order to acquire ultrasonic morphological volume image data, the time resolution and real time property of ultrasonic contrast volume image data deteriorate.

Accordingly, scanning conditions are set so that ultrasonic reflection echo signals for ultrasonic morphological image data can be received, in a B-mode scan, as the second reflected waves from an area or areas narrower than a 3D area which is a scanning area for a 3D contrast scan, i.e., a receiving target of the first reflected waves for generating ultrasonic contrast volume image data. In this case, it is practical to receive the second reflected waves, which are ultrasonic reflection echo signals for ultrasonic morphological image data, from an area or areas, whose a normal direction is an azimuth direction or an elevation direction, in a B-mode can.

The elevation direction is a moving direction of a scan plane. Meanwhile, the azimuth direction is an array direction of ultrasonic transducers in a direction parallel to a scan plane. Moreover, a plane parallel to a scan plane at the center position of the elevation direction is also called A side. Meanwhile, a plane orthogonal to the A side and parallel to the elevation direction is also called B side. Furthermore, a plane orthogonal to both the A side and the B side is called C side.

Therefore, in case of a mechanical 4D probe having one dimensionally arrayed ultrasonic transducers, the array direction of the ultrasonic transducers is the azimuth direction while the mechanically swinging direction of the mechanical 4D probe is the elevation direction. Meanwhile, in case of a 2D array probe whose ultrasonic transducers are arrayed two dimensionally, the array direction, in the direction parallel to a scan plane, out of the array directions of the ultrasonic transducers is the azimuth direction while the electronically swinging direction of a scan plane by the 2D array probe is the elevation direction.

Note that, the ultrasonic probe 3 can be classified into the convex type which has a sectorial scan plane formed by two arcs and two straight lines, the sector type which has a sectorial scan plane formed by one arc and two straight lines, and the linear type which has a rectangular scan plane. Therefore, the elevation direction becomes a straight line or an arc according to a type of the ultrasonic probe 3. Specifically, in case of a linear type, the elevation direction becomes a straight line. Meanwhile, in case of a convex type and a sector type, the elevation direction becomes an arc. Moreover, when the ultrasonic probe 3 is a convex type, ultrasonic transducers are arranged in an arc shape in the direction parallel to a scan plane. Therefore, in case of a convex type, an azimuth direction becomes an arc. Meanwhile, in case of a linear type and a sector type, an azimuth direction becomes a straight line.

In order to secure the frame rate of ultrasonic morphological image data with also securing the time resolution and the real time property of ultrasonic contrast volume image data, it is preferable to receive ultrasonic reflection echo signals (the second reflected waves) for ultrasonic morphological image data multiple times at timings such that a 3D area to be a target of 3D contrast scan (to be a receiving target of the first reflected waves) is divided into temporally equal durations. Specifically, in case of receiving ultrasonic reflection echo signals for ultrasonic wave morphological image data N (N is a natural number) times in the middle of 3D contrast scan, it is preferable to receive the ultrasonic reflection echo signals for the ultrasonic morphological image data N times at timings such that a 3D area which is the acquisition target of ultrasonic contrast volume image data is divided into N+1 3D areas each having an equal duration. Thereby, a frame rate of the ultrasonic morphological image data can be increased up to N times of a frame rate of the ultrasonic contrast volume image data.

Moreover, by receiving ultrasonic reflection echo signals for ultrasonic morphological image data from an area or areas temporally and spatially equally dividing a 3D target area of a 3D contrast scan, a time phase gap between the ultrasonic morphological image data and ultrasonic contrast volume image data can be minimized.

Therefore, in order to keep a satisfactory time resolution and real time property of ultrasonic contrast volume image data with minimizing a time phase gap between ultrasonic morphological image data and the ultrasonic contrast volume image data, it is preferable to receive ultrasonic reflection echo signals for the ultrasonic morphological image data from a center area which divides a 3D target area of a 3D contrast scan into two equal parts in an azimuth direction or an elevation direction.

Ultrasonic reflection echo signals for ultrasonic morphological image data can be acquired from a 2D area or a 3D area narrower than a scanning area of a 3D contrast scan. However, as shown in FIG. 2, what is necessary for an orientation of scanning positions is a 2D ultrasonic morphological image. Therefore, it is effective to acquire ultrasonic reflection echo signals for ultrasonic morphological image data from a 2D area to generate an ultrasonic morphological image for a monitor as a 2D cross-section image of a tissue, from a viewpoint of increasing the time resolution and real time property of ultrasonic contrast volume image data.

However, as shown in FIG. 2, it is preferable to generate ultrasonic morphological volume image data for VOI setting and VOI tracking for a TIC generation of ultrasonic contrast image data. However, an image quality required of ultrasonic morphological volume image data to be generated is sufficient so long as VOI setting and VOI tracking can be performed.

Accordingly, ultrasonic morphological volume image data can be generated using a part of ultrasonic reflection echo signals, acquired from a 3D area, for ultrasonic contrast volume image data. Thereby, it is also possible to generate ultrasonic morphological volume image data for VOI setting and VOI tracking even when ultrasonic reflection echo signals for ultrasonic morphological image data are acquired from a 2D area in order to improve the time resolution and the real time property of ultrasonic contrast volume image data. That is, it is possible to satisfy all of increase of the time resolution and the real time property of ultrasonic contrast volume image data, generation of ultrasonic morphological images with a high quality for monitor, and generation of ultrasonic morphological volume image data for VOI setting and VOI tracking.

Therefore, in order to gain the most satisfactory time resolution and real time property of ultrasonic contrast volume image data, a scan sequence for receiving ultrasonic reflection echo signals (the second reflected waves) for ultrasonic morphological image data from a 2D area which divides a target 3D area of a 3D contrast scan (which is a receiving target of the first reflected waves) into two parts and whose normal direction is an azimuth direction or an elevation direction is practical and preferable conditions.

In this case, the 2D area, which is an acquisition target of ultrasonic reflection echo signals for ultrasonic morphological image data, is on the A side at the center position in the elevation direction or the B side at the center position in the azimuth direction. Therefore, an ultrasonic morphological image displayed for a monitor is also an image of the A side direction or the B side direction.

FIG. 3 is a view showing the first example of a scan sequence for performing a B-mode scan in the middle of a 3D contrast scan.

In FIG. 3, the arc direction parallel to the paper shows an elevation direction of the ultrasonic probe 3. Moreover, in FIG. 3, the solid lines show positions of scan planes in the 3D contrast scan while the dotted line shows a position of scan plane in the B-mode scan. However, the solid line and the dotted line which actually overlap are described without overlapping them for simplified explanation.

As shown in FIG. 3, a scan sequence can be determined so that ultrasonic reflection echo signals for ultrasonic contrast volume image data are acquired sequentially from scan planes in the elevation direction. Furthermore, the scan sequence can be determined so that ultrasonic reflection echo signals for ultrasonic morphological image data can be acquired from the 2D area which divides a 3D target area of the 3D contrast scan into two equal parts in the elevation direction, i.e., the A side at the center position of the 3D area.

Therefore, when the number of the scan planes for the 3D contrast scan is odd, the scan plane for the B-mode scan overlaps with the central scan plane for the 3D contrast scan. Moreover, in terms of time, the B-mode scan is performed at the central time of the 3D contrast scan. Thereby, a time phase gap between the ultrasonic reflection echo signals for the ultrasonic contrast volume image data acquired by the 3D contrast scan and the ultrasonic reflection echo signals for the ultrasonic morphological image data acquired by the B-mode scan can be minimized.

Moreover, it is possible to increase the time resolution and the real time property of the ultrasonic contrast volume image data by setting the B-mode scan as a 2D scan.

FIG. 4 is a schematic diagram showing an example of waveforms of transmission signals respectively transmitted in the 3D contrast scan and the B-mode scan shown in FIG. 3.

FIG. 4 (A) shows an example of waveforms of transmission signals transmitted in the 3D contrast scan while FIG. 4 (B) shows an example of a waveform of a transmission signal transmitted in the B-mode scan. As shown in FIG. 4 (A), the 3D contrast scan for all scan planes including the center scan plane and each scan plane other than the center plane can be performed by a three rate AM (amplitude modulation) method which modulates an amplitude and transmits a transmission signal having a low frequency three times, for example. On the other hand, as shown in FIG. 4 (B), the B-mode scan can be performed only for the center scan plane in the one rate fundamental wave mode which transmits a transmission signal, whose amplitude and frequency are not modulated, in the fundamental wave band, one time.

Thus, a scan sequence for an alternate scan which performs the 3D contrast scans and the B-mode scans alternately can be set in units of scan plane in the scan control part 10, and each B-mode scan can be performed in the middle of a 3D contrast scan according to the set scan sequence.

Note that, as methods of a contrast scan besides the AM method, the PM method which modulates a phase and the AMPM (amplitude phase modulation) method which modulates both an amplitude and a phase are known as mentioned above. Thus, conditions including the method of a contrast scan and a transmission rate can be selected arbitrarily.

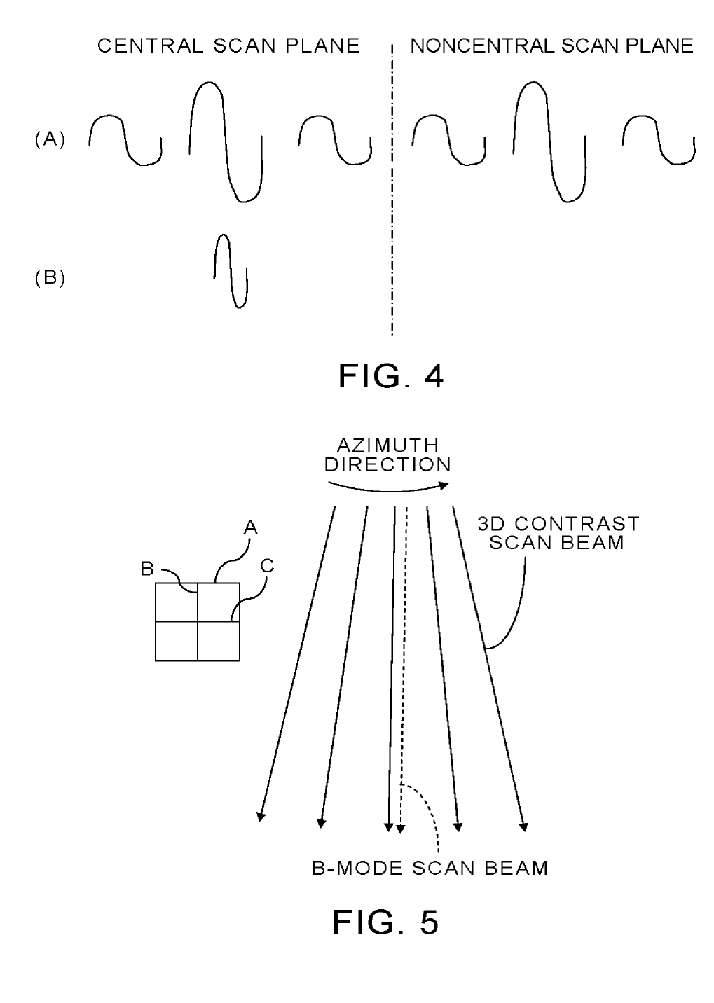

FIG. 5 is a view showing the second example of a scan sequence for performing a B-mode scan in the middle of a 3D contrast scan.

In FIG. 5, the arc direction parallel to the paper shows an azimuth direction of the ultrasonic probe 3. Moreover, in FIG. 5, the solid lines show positions of transmission beams in the 3D contrast scan while the dotted line shows a position of transmission beam in the B-mode scan. However, the solid line and the dotted line which actually overlap are described without overlapping them for simplified explanation.

As shown in FIG. 5, a scan sequence can be determined so that ultrasonic reflection echo signals for the ultrasonic contrast volume image data are acquired sequentially from B sides in the azimuth direction. Furthermore, the scan sequence can be determined so that ultrasonic reflection echo signals for the ultrasonic morphological image data can be acquired from the 2D area which divides the 3D target area of the 3D contrast scan into two equal parts in the azimuth direction, i.e., the B side at the center position of the 3D area.

In this case, transmission beams are transmitted toward scanning positions, which are different in the azimuth direction, on each scan plane, by the 3D contrast scan. Meanwhile, a transmission beam is transmitted only to the center position in the azimuth direction, on each scan plane, by the B-mode scan.

Therefore, when the number of B sides which are targets of the 3D contrast scan is odd, the B side for the B-mode scan overlaps with the center one of the B sides which are targets of the 3D contrast scan. Moreover, in terms of time, when the transmission beams for the 3D contrast scan are transmitted sequentially, the transmission beam for the B-mode scan is transmitted at the center time in the transmission period of the transmission beams for the 3D contrast scan, on each scan plane. Thereby, the time phase gap between ultrasonic reflection echo signals, for the ultrasonic contrast volume image data, acquired by the 3D contrast scan and ultrasonic reflection echo signals, for the ultrasonic morphological image data, acquired by the B-mode scan can be minimized.

Moreover, it is possible to increase the time resolution and the real time property of the ultrasonic contrast volume image data since the B-mode scan acquires ultrasonic reflection echo signals from a 2D area.

FIG. 6 is a schematic diagram showing an example of waveforms of transmission signals respectively transmitted in the 3D contrast scan and the B-mode scan shown in FIG. 5.

FIG. 6 (A) shows an example of waveforms of transmission signals transmitted in the 3D contrast scan while FIG. 6 (B) shows an example of a waveform of a transmission signal transmitted in the B-mode scan. As shown in FIG. 6 (A), the 3D contrast scan can be performed by transmitting ultrasonic transmission beams to the entire target area in the azimuth direction by a three rate AM method, for example. On the other hand, as shown in FIG. 6 (B), the B-mode scan can be performed by the one rate fundamental wave mode which transmits an ultrasonic transmission beam consisting of transmission signals, in the fundamental wave band, whose amplitude and frequency are not modulated, only to the center position in the azimuth direction, one time.

Thus, a scan sequence for an alternate scan which performs the 3D contrast scans and the B-mode scans alternately can be set in units of scan plane in the scan control part 10, and each B-mode scan can be performed in the middle of a 3D contrast scan according to the set scan sequence.

As a matter of course, a scan sequence can also be set so that ultrasonic reflection echo signals for ultrasonic morphological image data on two cross-sections orthogonal to each other can be acquired from both the A side and the B side. In that case, what is necessary is to combine the scan sequence exemplified in FIG. 3 with the scan sequence exemplified in FIG. 5. Moreover, a scan sequence can also be set so that ultrasonic reflection echo signals for ultrasonic morphological image data can be acquired from a plane or planes which are parallel to neither the A side nor the B side.

That is, it is possible to set a scan sequence which performs a 3D contrast scan for acquiring ultrasonic reflection echo signals for ultrasonic contrast volume image data from a 3D area and a B-mode scan for acquiring ultrasonic reflection echo signals for ultrasonic morphological image data from a specific scan plane or specific scan planes in the elevation direction and a specific scanning position or specific scanning positions in the azimuth direction.

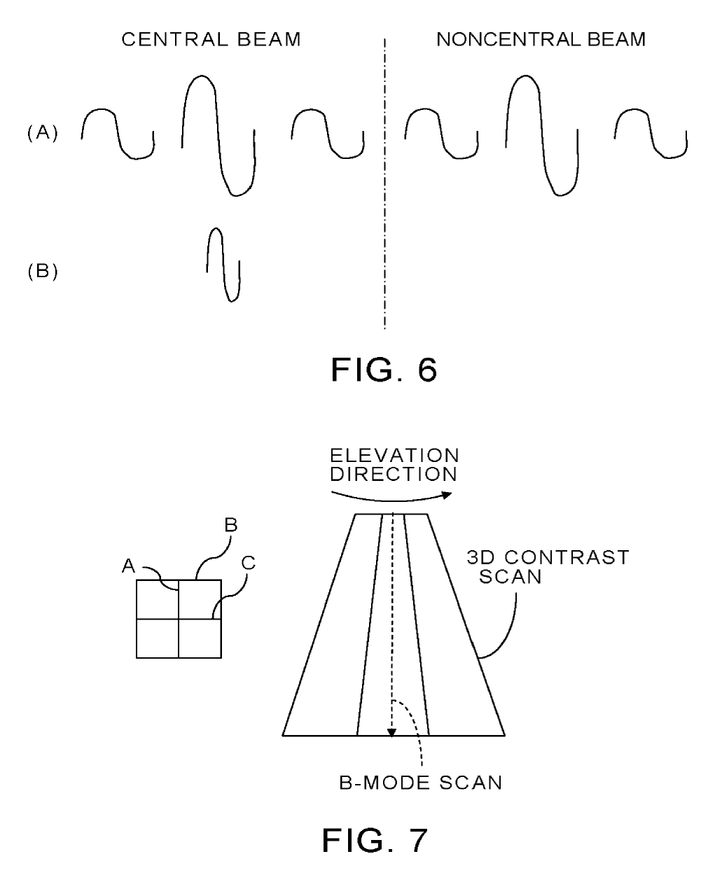

FIG. 7 is a view showing the third example of a scan sequence for performing a B-mode scan in the middle of a 3D contrast scan.

In FIG. 7, the arc direction parallel to the paper shows the elevation direction of the ultrasonic probe 3. Moreover, in FIG. 7, a dotted line shows a position of a scan plane for the B-mode scan while the respective areas surrounded by the solid lines show 3D target areas, for the 3D contrast scan, divided temporally by repeating the B-mode scan multiple times.

As shown in FIG. 7, a scan sequence can be determined so that ultrasonic reflection echo signals for ultrasonic contrast volume image data are acquired sequentially from scan planes in the elevation direction. Furthermore, the scan sequence can be determined so that ultrasonic reflection echo signals for ultrasonic morphological image data can be acquired multiple times, during the 3D contrast scan, from the 2D area which divides the 3D target area of the 3D contrast scan into two parts in the elevation direction. In the example shown in FIG. 7, the 3D contrast volume area which is a target of the 3D contrast scan is divided into three spatially equal parts in the elevation direction by the B-mode scan two times at the center position of the 3D contrast volume area.

That is, a scan sequence can also be determined so that ultrasonic reflection echo signals for ultrasonic morphological image data are acquired multiple times during a 3D contrast scan. When ultrasonic reflection echo signals for ultrasonic morphological image data are acquired multiple times during a 3D contrast scan, the frame rate of ultrasonic morphological images can be increased.

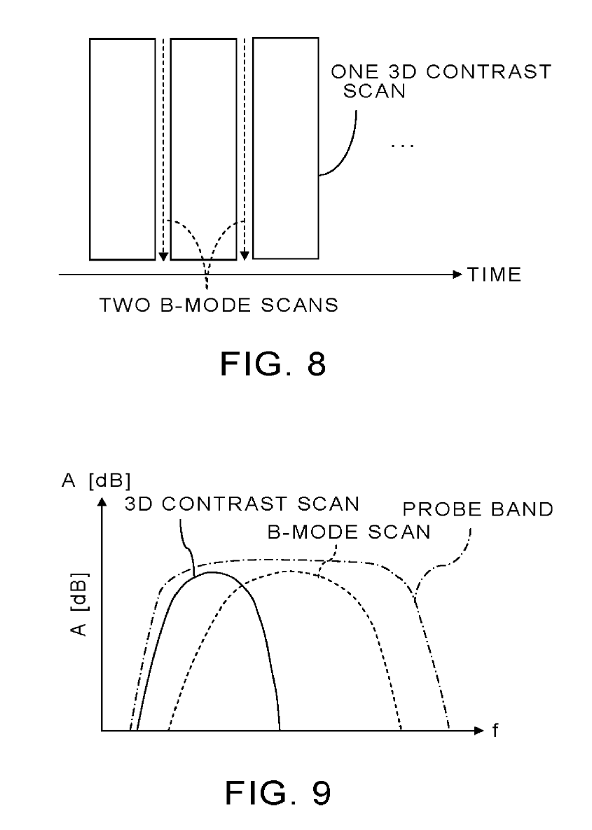

FIG. 8 is a view showing each update rate of the ultrasonic morphological image data acquired by the B-mode scan and the ultrasonic contrast volume image data acquired by the 3D contrast scan, shown in FIG. 7.

In FIG. 8, the horizontal axis shows time while the direction of the vertical axis shows a direction parallel to a scan plane. As shown in FIG. 8, scanning timings to the segmented 3D areas, which are the targets of the 3D contrast scan, and scanning timings of the B-mode scans to the center position of the 3D contrast volume, shown in FIG. 7, are indicated in the time direction. That is, when a B-mode scan is performed multiple times during a 3D contrast scan, the update rate of ultrasonic morphological image data becomes higher than the update rate of ultrasonic contrast volume image data.

Specifically, when a volume area which is a target of a 3D contrast scan is temporally and spatially divided into (N+1) areas, so that ultrasonic reflection echo signals for ultrasonic morphological image data can be acquired N times during the 3D contrast scan from the 2D area at the center position of a 3D contrast volume, the frame rate of the ultrasonic morphological image data becomes N times the update rate of ultrasonic contrast volume image data.

In the example shown in FIG. 7 and FIG. 8, the volume area which is a target of the 3D contrast scan is divided into three areas temporally and spatially by the two B-mode scans. In this case, the frame rate of the ultrasonic morphological image data is twice the update rate of the ultrasonic contrast volume image data. In other words, the ultrasonic morphological image data is updated twice while the ultrasonic contrast volume image data is updated once.

Thus, the frame rate of ultrasonic morphological image data can be set higher than the update rate of ultrasonic contrast volume image data so that an orientation of scanning positions can be performed effectively. In that case, a time phase gap between ultrasonic contrast volume image data and ultrasonic morphological image data can be minimized by repeating a B-mode scan multiple times at timings each temporally and spatially equally dividing a volume area which is a target of a 3D contrast scan.

Therefore, conditions of a B-mode scan may also be set as an update rate ratio of ultrasonic morphological image data to ultrasonic contrast volume image data as exemplified in FIG. 8. In particular, when an update rate ratio of ultrasonic morphological image data to ultrasonic contrast volume image data, i.e., a value obtained by dividing a frame rate of the ultrasonic morphological image data by an update rate of the ultrasonic contrast volume image data is N, scanning conditions to repeat a B-mode scan N times at timings, at which a target volume area of a 3D contrast scan is equally divided temporally and spatially in the elevation direction into (N+1) areas, can be set.

In this case, when N is set to 1, the frame rate of the ultrasonic morphological image data agrees with the update rate of the ultrasonic contrast volume image data. Therefore, the conditions for the alternate scan exemplified in FIG. 3 are set. Specifically, a scan sequence which acquires ultrasonic reflection echo signals for ultrasonic morphological image data from a single area set in a volume area which is a target of a 3D contrast scan is set.

Thus, the scan control part 10 can set receiving conditions of ultrasonic reflection echo signal (the second reflected waves) for ultrasonic morphological image data as a scan plane which is a target of a B-mode scan, scanning positions which are a target of a B-mode scan, or a frame rate of the ultrasonic morphological image data, such as 2D ultrasonic image data. Namely, the scan control part 10 can set a scan sequence which performs 3D contrast scans and B-mode scans alternately in units of a scan plane, a scan sequence which performs 3D contrast scans and B-mode scans alternately in units of a transmission beam, or a scan sequence which performs 3D contrast scans and B-mode scans alternately according to an update rate.

For that purpose, the scan control part 10 has a function to display a setting screen of scanning conditions on the display unit 7. Then, scanning conditions can be set by operation of the input device 8 with referring to the setting screen of scanning conditions. That is, the scan control part 10 has a function as a U/I (user interface) for setting scanning conditions.

The setting screen of scanning conditions allows setting conditions, such as setting a target area of a B-mode scan to either one of only a 2D area perpendicular to the elevation direction, only a 2D area perpendicular to the azimuth direction, or both of a 2D area perpendicular to the elevation direction and a 2D area perpendicular to the azimuth direction. The setting screen of scanning conditions also allows setting an update rate ratio of ultrasonic morphological image data to ultrasonic contrast volume image data.

Moreover, the scan control part 10 can make it possible to adjust the frame rate of ultrasonic morphological image data, such as 2D ultrasonic image data, according to direction information input from the input device 8. Specifically, the number of receptions of ultrasonic reflection echo signals for ultrasonic morphological image data during one 3D contrast scan for a volume area can be adjusted manually. In this case, an update rate ratio of ultrasonic morphological image data to ultrasonic contrast volume image data may be manually adjusted by operation of the input device 8.

The scan control part 10 also sets a frequency band of transmission signals applied to the ultrasonic probe 3, besides a volume area to be a target of a 3D contrast scan and a target area of a B-mode scan, as described above. The frequency band of transmission signals can be set in a wide band so that a sensitivity of ultrasonic reflection signals reflected on a contrast agent becomes satisfactory while ultrasonic morphological images can be obtained in a high image quality. However, setting a frequency band of transmission signals in a more appropriate band according to whether a part of reception signals acquired by a 3D contrast scan is used for generation of ultrasonic morphological volume image data leads to an effective use of energy.

FIG. 9 is a graph showing appropriate frequency characteristics of transmission signals when reception signals acquired by a 3D contrast scan are used only for generation of ultrasonic contrast volume image data.

In FIG. 9, the horizontal axis shows frequencies f while the vertical axis shows amplitudes A [dB] of transmission signals. The respective frequency bands of transmission signals for a 3D contrast scan and a B-mode scan can be individually and respectively set within a range of frequency band of transmission signals, which can be applied to the ultrasonic probe 3, as shown by the dashed-dotted line in FIG. 9.

The frequency band of transmission signals for a 3D contrast scan can be set in a low-frequency region, where the sensitivity of ultrasonic reflection signals reflected on an ultrasonic contrast agent becomes satisfactory, as shown by the solid line in FIG. 9. Meanwhile, the frequency band of transmission signals for a B-mode scan for acquiring ultrasonic morphological image data for a monitor can be set in a wide band, around the center of the frequency band of transmission signals which can be applied to the ultrasonic probe 3, as shown by the dotted line in FIG. 9, so that an image quality becomes satisfactory.

That is, ultrasonic waves, which have a frequency characteristic appropriate for generation of ultrasonic contrast volume image data, can be transmitted respectively as the first ultrasonic waves and ultrasonic waves, obtained by modulating the amplitude and/or the phase of the first ultrasonic waves with a predetermined ratio, from the ultrasonic probe 3 in a 3D contrast scan while ultrasonic waves, which have a frequency characteristic appropriate for generation of ultrasonic morphological image data, can be transmitted as the second ultrasonic waves from the ultrasonic probe 3 in a B-mode scan when reception signals acquired by the 3D contrast scan are used only for the generation of the ultrasonic contrast volume image data. Thus, the energy efficiency for generating transmission signals can be raised by setting the frequency characteristic of the transmission signals to one appropriate for every scan.

On the other hand, it is effective to transmit ultrasonic waves, which have a frequency characteristic appropriate for generation of both ultrasonic morphological volume data and ultrasonic contrast image data, from the ultrasonic probe 3, respectively as the first ultrasonic waves and ultrasonic waves, obtained by modulating the amplitude and/or the phase of the first ultrasonic waves with a predetermined ratio, in a 3D contrast scan when reception signals acquired by the 3D contrast scan are used for the generation of both the ultrasonic contrast volume image data and the ultrasonic morphological volume image data.

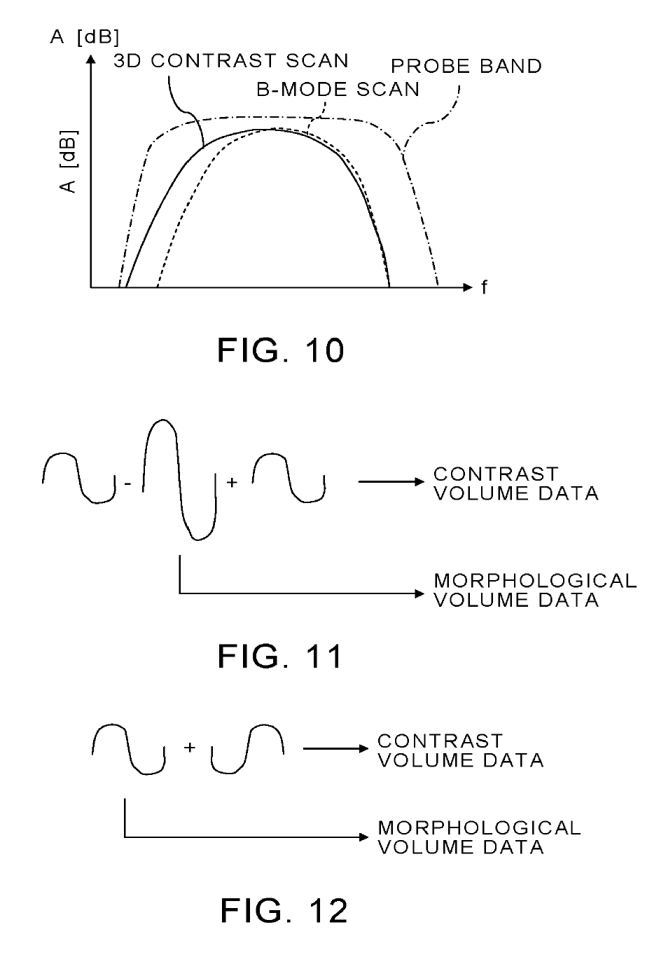

FIG. 10 is a graph showing appropriate frequency characteristics of transmission signals when a part of reception signals acquired by a 3D contrast scan are used for generation of ultrasonic morphological volume image data.

In FIG. 10, the horizontal axis shows frequencies f while the vertical axis shows amplitudes A [dB] of transmission signals. As is the case with an example shown in FIG. 9, the respective frequency bands of transmission signals for a 3D contrast scan and a B-mode scan can be individually and respectively set within a range of frequency band of transmission signals, which can be applied to the ultrasonic probe 3, as shown by the dashed-dotted line in FIG. 10.

As is the case with an example shown in FIG. 9, the frequency band of transmission signals for a B-mode scan can be set in a wide band, around the center of the frequency band of transmission signals which can be applied to the ultrasonic probe 3, as shown by the dotted line in FIG. 10, so that an image quality becomes satisfactory.

On the other hand, the frequency band of transmission signals for a 3D contrast scan can be set to a wider band including a low-frequency region, where the sensitivity of ultrasonic reflection signals reflected with a contrast agent becomes satisfactory, and the vicinity of the center of the frequency band of transmission signals which can be applied to the ultrasonic probe 3, as shown by the solid line in FIG. 10, so that ultrasonic morphological images of a tissue can be obtained in a high image quality. That is, transmission signals for a 3D contrast scan can be wide band signals preferable for imaging both a contrast agent and a tissue.

Other scanning conditions set in the scan control part 10 include conditions for scanning by the parallel simultaneous receiving method. The parallel simultaneous receiving method is a technique that divides ultrasonic transducers included in the ultrasonic probe 3 into ultrasonic transducer groups in order to receive ultrasonic echo signals simultaneously from different raster directions by controlling every ultrasonic transducer group independently. When scanning is performed by the parallel simultaneous receiving method, a plane wave or a diffusion wave is transmitted toward the object P, as a transmission pattern of ultrasonic waves.

When scanning conditions are set so that at least one of a 3D contrast scan and a B-mode scan is performed by the parallel simultaneous receiving method which receives ultrasonic waves simultaneously from mutually different directions using plural ultrasonic transducers, the time resolution and real time property of ultrasonic contrast volume image data can be increased further, and also the time phase gap between ultrasonic morphological image data and the ultrasonic contrast volume image data can be reduced.

Especially, it is also possible to receive ultrasonic reflection signals for ultrasonic morphological image data and ultrasonic reflection signals for ultrasonic contrast volume image data almost simultaneously from scanning positions on a same scan plane. That is, it is also possible to transmit and receive ultrasonic signals by a B-mode scan during transmission and reception of ultrasonic signals by a 3D contrast scan, under the parallel simultaneous receiving method.

Next, functions of the data processing system 5 will be described.

The data processing system 5 has a function to generate ultrasonic contrast volume image data based on the first reception signals acquired as volume data by a 3D contrast scan performed as the first scan; and to generate ultrasonic morphological image data for a monitor based on the second reception signals acquired by a B-mode scan performed as the second scan. In addition, the data processing system 5 is configured to be able to generate ultrasonic morphological volume data, where a form of the object P has been depicted, based on a part of the first reception signals acquired for generation of ultrasonic contrast volume image data.

The Doppler processing part 12 of the data processing system 5 has a function to acquire the first reception signals, acquired as ultrasonic Doppler signals from a volume area by a 3D contrast scan, from the transceiver unit 9, and to generate ultrasonic contrast volume image data which displays dynamic information, including a velocity, a dispersion and a power of a blood flow, with colors or the like, by performing Doppler processing including frequency analysis. Specifically, the Doppler processing part 12 has a function as a signal processing part which generates composite signals by combining the first reception signals based on the first reflected waves received by a 3D contrast scan which is performed as the first scan, based on a predetermined ratio used for modulation of at least one of the amplitude and the phase in the 3D contrast scan, and a function as an image generation part which generates 3D ultrasonic image data as ultrasonic contrast volume image data based on the composite signals.

Note that, discontinuous lines may be depicted at positions corresponding to division positions of a volume area which is a target of a 3D contrast scan when ultrasonic contrast volume image data is displayed by 2D display processing. Accordingly, in order to reduce a discontinuity in a displayed contrast image, a smoothing filter may be applied to ultrasonic contrast volume image data in the Doppler processing part 12. In that case, it is appropriate to apply a smoothing filter locally only to positions corresponding to division positions of a volume area since the division positions of the volume area are known information set as scanning conditions.

The B-mode processing part 11 has a function to acquire the second reception signals, acquired by a B-mode scan, from the transceiver unit 9, and to generate ultrasonic morphological image data, as B-mode image data where intensities of the second reception signals are displayed with brightness, by performing generation processing of the B-mode image data including logarithmic conversion processing and envelope detection processing. That is, the B-mode processing part 11 has a function as an image generation part which generates ultrasonic morphological image data, such as 2D ultrasonic image data, based on the second reception signals on the basis of the second reflected waves received by a B-mode scan performed as the second scan.

Moreover, the B-mode processing part 11 has a function to acquire the first reception signals, acquired from a volume area by a 3D contrast scan, from the transceiver unit 9, and to generate ultrasonic contrast volume image data for displaying a distribution of a contrast agent with a gray scale. Similarly in this case, a smoothing filter is applicable in order to reduce discontinuities at division positions of a volume area which is a target of a 3D contrast scan.

Furthermore, the B-mode processing part 11 has a function to generate ultrasonic morphological volume data based on a part of the first reception signals acquired by a 3D contrast scan.

The reception signals acquired by a 3D contrast scan include nonlinear component generated by being reflected to a contrast agent and linear component generated by being reflected to a tissue. Therefore, when a linear operation is performed among reception signals acquired by sequentially transmitting ultrasonic waves modulated by a three rate AM method, as exemplified in FIG. 4 or FIG. 6, or another modulating method, linear component generated by being reflected to tissues can be removed in order to extract nonlinear component generated by being reflected to a contrast agent. Then, it is possible to generate ultrasonic contrast volume image data using the extracted nonlinear component.

However, reception signals, before the linear operation, acquired by a 3D contrast scan include linear component generated by being reflected to tissues. Accordingly, ultrasonic morphological volume image data can be generated using the linear component included in reception signals, before the linear operation, acquired from a 3D volume area by a 3D contrast scan.

FIG. 11 is a view describing a method of generating ultrasonic contrast volume image data and ultrasonic morphological volume data by a 3D contrast scan under a three rate AM method.

In a case of simple three rate AM method, an ultrasonic wave whose amplitude has been modulated twice and two ultrasonic waves whose amplitudes are not modulated, as shown in FIG. 11, are transmitted sequentially in a 3D contrast scan. Therefore, linear components from tissues can be cancelled to extract nonlinear component from a contrast agent by a linear operation which subtracts a reception signal, acquired by transmission of the ultrasonic wave whose amplitude has been modulated twice, from two reception signals acquired by transmission of the two ultrasonic waves whose amplitudes are not modulated. Thus, it is possible to generate ultrasonic contrast volume image data which shows blood flow dynamic information, including a velocity, a power and a dispersion of a blood flow, by data processing including Doppler analysis of the extracted nonlinear component. Alternatively, it is also possible to generate ultrasonic contrast volume image data, which shows a distribution of a contrast agent in a gray scale, by simple processing.

On the other hand, each of the three reception signals before the linear operation includes the linear component and the nonlinear component. Accordingly, the linear component can be extracted from one reception signal by filter processing in a frequency direction or the like. Since reception signals are acquired from a 3D volume area by a 3D contrast scan, ultrasonic morphological volume image data can be generated based on the linear components of the reception signals acquired from the 3D area.

FIG. 12 is a view describing a method of generating ultrasonic contrast volume image data and ultrasonic morphological volume image data by a 3D contrast scan under a PM method.

In a case of simple PM method, two ultrasonic waves, of which a phase of one wave has been inverted, as shown in FIG. 12, are transmitted sequentially in a 3D contrast scan. Therefore, linear components from tissues can be cancelled to extract nonlinear component from a contrast agent by a linear operation adding two reception signals corresponding to the two ultrasonic transmission waves. Thus, ultrasonic contrast volume image data is generable by data processing of the extracted nonlinear component.

On the other hand, each of the two reception signals before the linear operation includes linear component and nonlinear component. Accordingly, linear component can be extracted from one reception signal by filter processing in a frequency direction or the like. Since reception signals are acquired from a 3D volume area by a 3D contrast scan, ultrasonic morphological volume image data can be generated based on linear components of the reception signals acquired from the 3D area.

As described above, the B-mode processing part 11 has a function to obtain a part of reception signals, acquired by a 3D contrast scan, from the transceiver unit 9, and to generate ultrasonic morphological volume image data as B-mode image data by extracting linear components from the part of the acquired reception signals.

The display processing part 13 has a function to perform necessary display processing of ultrasonic morphological image data acquired from the B-mode processing part 11 and ultrasonic contrast volume image data acquired from the Doppler processing part 12, in order to display them on the display unit 7 as 2D morphological images and 2D contrast images. Examples of the display processing include filter processing for determining an image quality, scanning conversion to convert image signals in a scanning line format into image signals in a video format, and 2D conversion processing of ultrasonic volume image data. Examples of the 2D conversion processing for generating 2D image data for a display from 3D volume image data include VR (Volume Rendering) processing, MIP (Maximum Intensity Projection) processing and MPR (Multi Planer Reconstruction) processing.

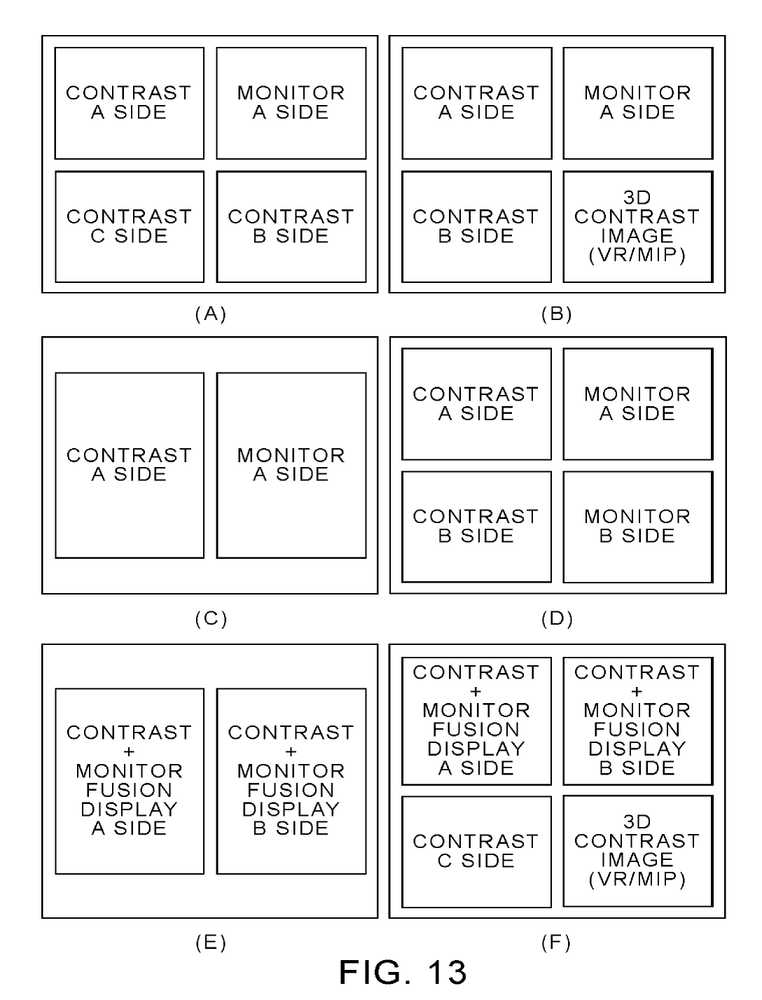

FIG. 13 is a view showing a variation of methods for displaying ultrasonic morphological images for monitor and contrast images.

The display processing part 13 can perform display processing so that 2D morphological images for a monitor and 2D contrast images can be displayed on the display unit 7 with various layouts. The views from (A) to (F) in FIG. 13 show examples of layout for 2D morphological images for monitor and 2D contrast images.

Specifically, (A) is a layout which displays a contrast image in the A side direction, a contrast image in the B side direction, a contrast image in the C side direction, and a morphological image for a monitor in the A side direction, in parallel. (B) is a layout which displays a contrast image in the A side direction, a contrast image in the B side direction, a morphological image for a monitor in the A side direction, and a 3D contrast image, in parallel. (C) is a layout which displays a contrast image in the A side direction and a morphological image for a monitor in the A side direction, in parallel. These layouts from (A) to (C) that display a monitor image only in the A side direction are appropriate when a B-mode scan has been performed with setting a 2D scanning area only in the A side direction.

Note that, a VR image or an MIP image may be displayed as a 3D image. Moreover, a 2D contrast image in a desired cross-section, such as the A side direction, the B side direction, or the C side direction, can be generated for a display by MPR processing based on ultrasonic volume image data.

(D) is a layout which displays a contrast image in the A side direction, a contrast image in the B side direction, a morphological image for a monitor in the A side direction, and a morphological image for a monitor in the B side direction, in parallel. (E) is a layout which displays an A side image consisting of a superimposed contrast image and morphological image for a monitor in the A side direction, and a B side image consisting of a superimposed contrast image and morphological image for a monitor in the B side direction, in parallel. (F) is a layout which displays an A side image consisting of a superimposed contrast image and morphological image for a monitor in the A side direction, a B side image consisting of a superimposed contrast image and morphological image for a monitor in the B side direction, a contrast image in the C side direction, and a 3D contrast image, in parallel.

These layouts from (D) to (F) that display monitor images in both the A side direction and the B side direction can be adopted when B-mode scans have been performed with setting 2D scanning areas in both the A side direction and the B side direction. Especially, sizes of displayed images can be enlarged when contrast images are superimposed and displayed on morphological images. Thereby, reduction in visibility is avoidable with securing an amount of information to be displayed.

A layout of displayed images exemplified in FIG. 13 can be arbitrarily set by inputting direction information on an image to be a display target, from the input device 8 to the display processing part 13. That is, the display processing part 13 also has a function as a U/I for setting a layout of displayed images.

Moreover, even in case of a same examination, a layout can be changed according to an observing situation of images. That is, images to be display targets can be changed. For example, a superimposed image in the A side direction and a superimposed image in the B side direction can be displayed in parallel as shown in (E) when contrast images are observed in real time during a scan while a contrast image in the C side direction and a 3D contrast image can be displayed additionally as shown in (F) in case of an observation after the freeze.

Besides the foregoing display processing of image data, the display processing part 13 has a function to perform a TIC analysis of ultrasonic contrast volume image data. When a TIC is generated, a VOI which is a generation target of the TIC is set. In order to set a VOI which is a generation target of a TIC, it is favorable to use ultrasonic morphological volume image data, generated in the B-mode processing part 11, as a reference image. When a respiratory or pulsatility motion or movement exists in an interesting part, a position of VOI is corrected. That is, VOI tracking is performed. In that case, it is realistic to refer to ultrasonic morphological volume image data from a viewpoint of keeping a tracking accuracy.

The storage unit 6 can store ultrasonic morphological image data and ultrasonic contrast volume image data which have been obtained by the display processing part 13. Especially, when ultrasonic morphological volume image data have been obtained in the display processing part 13, the ultrasonic morphological volume image data can be stored in the storage unit 6 with relating the ultrasonic morphological volume image data to corresponding ultrasonic contrast volume image data. Thus, the display processing part 13 is configured to be able to read arbitrary ultrasonic image data, such as ultrasonic morphological volume image data, which have been stored in the storage unit 6, to use the read ultrasonic image data as a target of display processing or a TIC analysis.

Out of the above mentioned elements of the apparatus main body 2, elements which process digital information can be configured by a computer reading program for the ultrasonic diagnostic apparatus 1. However, circuits may be used to configure an arbitrary element of the apparatus main body 2.

Specifically, a computer can function as the transceiver part 4 by reading control program of the ultrasonic diagnostic apparatus 1. In addition, a computer can function as the data processing system 5 by reading data processing program of the ultrasonic diagnostic apparatus 1.

A program including control program and data processing program of the ultrasonic diagnostic apparatus 1 can be recorded on an information recording medium to be distributed as a program product. Therefore, an existing ultrasonic diagnostic apparatus can function as the ultrasonic diagnostic apparatus 1 shown in FIG. 1 by installing necessary program into the existing ultrasonic diagnostic apparatus.

Next, an operation and an action of the ultrasonic diagnostic apparatus 1 will be explained.

Firstly, a scanning mode, which performs a B-mode scan for acquiring ultrasonic morphological image data for a monitor, during a 3D contrast scan for acquiring time series ultrasonic contrast volume image data in real-time, is selected by operation of the input device 8 through a setting screen of scanning conditions displayed, on the display unit 7, by the scan control part 10.

Next, at least one area which is a target of a B-mode scan is set as an area narrower than a volume area which is a target of a 3D contrast scan. Preferably, at least one 2D area is set as an area which is a target of a B-mode scan. In that case, the number, positions and directions of 2D areas which are targets of B-mode scans are set. Specifically, each position of 2D area which is a target of a B-mode scan or an update rate ratio of ultrasonic morphological image data to ultrasonic contrast volume image data can be set. As a result, scanning areas to be targets of 3D contrast scans and B-mode scans are set as exemplified in FIG. 3, FIG. 5, or FIG. 7.

In addition, it is determined whether to generate ultrasonic morphological volume image data using a part of reception signals acquired by a 3D contrast scan. In case of generating no ultrasonic morphological volume image data, a frequency characteristic of transmission signals as exemplified in FIG. 9 can be set as a scanning condition. Specifically, scanning conditions can be set so that ultrasonic waves in a low frequency band are transmitted in a 3D contrast scan in order to receive ultrasonic reflection signals from a contrast agent with satisfactory sensitivity while ultrasonic waves in a wide band near the center of a frequency band of transmission signals, which can be applied to the ultrasonic probe 3, are transmitted in a B-mode scan in order to obtain tissue cross-sectional images with a high image quality.

On the contrary, in case of generating ultrasonic morphological volume image data, a frequency characteristic of transmission signals as exemplified in FIG. 10 can be set as a scanning condition. Specifically, a wide frequency band including both a low frequency area and the vicinity of the center of a frequency band of transmission signals which can be applied to the ultrasonic probe 3 can be set as a frequency band of transmission signals in a 3D contrast scan so that ultrasonic reflection signals from a contrast agent can be received with satisfactory sensitivity while tissue cross-sectional images can be generated with a required image quality. Meanwhile, a band near the center of a frequency band of transmission signals which can be applied to the ultrasonic probe 3 can be set as a frequency band of transmission signals in a B-mode scan so that tissue cross-sectional images can be obtained with a high image quality.

However, a wider frequency band including both a low frequency area and a band near the center of a frequency band of transmission signals which can be applied to the ultrasonic probe 3 may be set as a frequency band common to respective transmission signals for a 3D contrast scan and a B-mode scan, regardless of whether to generate ultrasonic morphological volume image data.

As other scanning conditions, data acquisition methods, such as a modulation method of transmission signals in a 3D contrast scan, and conditions, such as whether parallel simultaneous receiving is performed, can be set. For example, transmission signals as exemplified in FIG. 4 or FIG. 6 are set as scanning conditions in case of performing a 3D contrast scan under a three rate AM method.

Next, a display layout of ultrasonic images acquired by 3D contrast scans and B-mode scans is set. A display layout of ultrasonic images can be selected from various alternatives as exemplified in FIG. 13. Specifically, when designating information for a display layout is input into the display processing part 13 by an operation of the input device 8, the display processing part 13 sets the display layout according to the designating information.

Next, an ultrasonic contrast agent, such as microbubbles, is injected into a blood vessel of the object P. Then, 3D contrast scans and B-mode scans are performed according to set scanning conditions. Specifically, an electric signal with a predetermined delay time is applied as a transmission signal to each ultrasonic transducer of the ultrasonic probe 3 from the transceiver unit 9 under control by the scan control part 10. As a result, an ultrasonic beam toward a scanning position in the object P is formed by ultrasonic signals transmitted from the respective ultrasonic transducers.

Then, ultrasonic reflection signals arise by reflection of ultrasonic waves at the scanning position. The ultrasonic reflection signals which have arisen at the scanning position are received with predetermined delay times by respective ultrasonic transducers of the ultrasonic probe 3. The ultrasonic reflection signals received by the respective ultrasonic transducers are converted into reception signals which are electric signals. Subsequently, the reception signals are output to the transceiver unit 9. In the transceiver unit 9, necessary signal processing including A/D conversion processing and phasing addition processing is performed. Thereby, ultrasonic reception data corresponding to each scanning position are generated.

Such acquisitions of ultrasonic reception data sets corresponding to scanning positions are performed sequentially by 3D contrast scans and B-mode scans according to scanning conditions. Accordingly, ultrasonic reception data sets corresponding to respective scanning positions in a volume area are acquired sequentially by a 3D contrast scan. In addition, ultrasonic reception data sets corresponding to respective scanning positions in a target area are acquired sequentially by a B-mode scan during a 3D contrast scan.

When a Doppler mode is selected as an image processing condition for a 3D contrast scan, the ultrasonic reception data acquired by the 3D contrast scan are output sequentially from the transceiver unit 9 to the Doppler processing part 12 as ultrasonic Doppler signals. Accordingly, the Doppler processing part 12 generates ultrasonic Doppler image data which show a blood flow dynamic state, including a velocity, a dispersion and a power of a blood flow in a volume area, by Doppler processing based on the ultrasonic Doppler signals. The generated ultrasonic Doppler image data are given to the display processing part 13 as ultrasonic contrast volume image data.

Alternatively, when a B-mode is selected as an image processing condition for a 3D contrast scan, the ultrasonic reception data acquired by the 3D contrast scan are output sequentially to the B-mode processing part 11. Accordingly, the B-mode processing part 11 generates ultrasonic contrast volume image data, where a distribution of the contrast agent in a volume area has been depicted, by B-mode processing based on the ultrasonic reception data acquired by the 3D contrast scan. The generated ultrasonic contrast volume image data are given to the display processing part 13.

Meanwhile, ultrasonic reception data acquired by a B-mode scan are output from the transceiver unit 9 to the B-mode processing part 11. Accordingly, the B-mode processing part 11 generates ultrasonic morphological image data, where a form of tissues in a scanning area has been depicted, by B-mode processing based on the ultrasonic reception data. The generated ultrasonic morphological image data are given to the display processing part 13.

Furthermore, when the scanning conditions have been set to generate ultrasonic morphological volume image data, the B-mode processing part 11 generates ultrasonic morphological volume image data, corresponding to a predetermined volume area, by B-mode processing based on a part of the ultrasonic reception data acquired by the 3D contrast scan. The generated ultrasonic morphological volume image data are given to the display processing part 13.

Therefore, when a 3D contrast scan and a B-mode scan are repeated, time series ultrasonic morphological image data and time series ultrasonic contrast volume image data are obtained in the display processing part 13. The display processing part 13 performs display processing sequentially based on the time series ultrasonic morphological image data and the time series ultrasonic contrast volume image data so that ultrasonic images can be displayed in a specified display layout. Then, the time series 2D image data generated as a result of the display processing are output sequentially to the display unit 7.

As a result, ultrasonic contrast images, where a blood flow dynamic state including a velocity, a dispersion and a power of a blood flow is displayed in color or the like, or ultrasonic contrast images, where a distribution of the contrast agent is depicted with a gray scale, are displayed with ultrasonic morphological images for a monitor, on the display unit 7, as moving images in real time, in a display layout as exemplified in FIG. 13. Therefore, a 3D contrast scan can be continued, with checking scanning positions with reference to ultrasonic morphological images where a form of tissues has been depicted.

Especially, ultrasonic signals for generating each ultrasonic morphological image have been acquired during a 3D contrast scan. Therefore, differences in time phase between ultrasonic morphological images for a monitor and ultrasonic contrast images are small. Consequently, scanning positions can be checked easily. Moreover, ultrasonic signals for generating each ultrasonic morphological image have been acquired from an area narrower than a volume area which is a target of a 3D contrast scan. Accordingly, it is possible to satisfactorily keep real time property of ultrasonic contrast images.

When ultrasonic morphological volume image data are generated, the ultrasonic morphological volume image data can be used as reference image data for VOI setting and for tracking the set VOI by position correction, in order to generate a TIC of ultrasonic contrast volume image data. Thereby, high-precision VOI setting and VOI tracking become possible.

That is, the ultrasonic diagnostic apparatus 1 as mentioned above is configured to perform a B-mode scan in the middle of a 3D contrast scan. Furthermore, the ultrasonic diagnostic apparatus 1 is configured to set a scanning area of a B-mode scan to an area, such as a 2D area at the center position in the A side direction or the B side direction, which is narrower than a scanning area of a 3D contrast scan.

Therefore, according to the ultrasonic diagnostic apparatus 1, differences in time phase between morphological image data for a monitor and ultrasonic contrast volume image data can be reduced, with keeping an update rate of the ultrasonic contrast volume image data. Thereby, an orientation of scanning positions can be performed accurately.

Furthermore, the ultrasonic diagnostic apparatus 1 can generate ultrasonic morphological volume image data using a part of reception signals acquired by a 3D contrast scan. Therefore, it becomes possible to perform VOI setting and tracking of the set VOI, for TIC generation of ultrasonic contrast volume image data, with a high precision. As a result, a TIC of ultrasonic contrast volume image data can be acquired for an appropriate VOI.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising:
a transceiver part configured to control an ultrasonic probe to perform:
a first scan which transmits a first ultrasonic wave and an ultrasonic wave, obtained by modulating an amplitude of the first ultrasonic wave, at least one time to each of scanning lines distributed three dimensionally, and receives first reflected waves due to transmissions of the first ultrasonic wave and the ultrasonic wave obtained by modulating the amplitude of the first ultrasonic wave; and
a second scan which transmits a second ultrasonic wave at least one time to each of scanning lines distributed two dimensionally during said first scan, and receives second reflective waves due to transmissions of the second ultrasonic wave;
said transceiver part obtaining first reception signals based on the first reflected waves and second reception signals based on the second reflected waves, from the ultrasonic probe;
a signal processing part configured to generate composite signals by combining the first reception signals; and
an image generation part configured to generate three dimensional ultrasonic image data based on the composite signals, and to generate two dimensional ultrasonic image data based on the second reception signals.

2. An ultrasonic diagnostic apparatus comprising:
a transceiver part configured to control an ultrasonic probe to perform:
a first scan which transmits a first ultrasonic wave and an ultrasonic wave, obtained by modulating a phase of the first ultrasonic wave, at least one time to each of scanning lines distributed three dimensionally, and receives first reflected waves due to transmissions of the first ultrasonic wave and the ultrasonic wave obtained by modulating the phase of the first ultrasonic wave; and
a second scan which transmits a second ultrasonic wave at least one time to each of scanning lines distributed two dimensionally during said first scan, and receives second reflective waves due to transmissions of the second ultrasonic wave;
said transceiver part obtaining first reception signals based on the first reflected waves and second reception signals based on the second reflected waves, from the ultrasonic probe;
a signal processing part configured to generate composite signals by combining the first reception signals; and
an image generation part configured to generate three dimensional ultrasonic image data based on the composite signals, and to generate two dimensional ultrasonic image data based on the second reception signals.

3. An ultrasonic diagnostic apparatus of claim 1,
wherein said transceiver part is configured to control the ultrasonic probe to perform a second scan which receives the second reflected waves from an area or each of areas narrower than a three dimensional area to be a receiving target of the first reflected waves.

4. An ultrasonic diagnostic apparatus of claim 2,
wherein said transceiver part is configured to control the ultrasonic probe to perform a second scan which receives the second reflected waves from an area or each of areas narrower than a three dimensional area to be a receiving target of the first reflected waves.

5. An ultrasonic diagnostic apparatus of claim 1,
wherein said transceiver part is configured to control the ultrasonic probe to perform a second scan which receives the second reflected waves from an area or each of areas whose normal direction is an azimuth direction or an elevation direction.

6. An ultrasonic diagnostic apparatus of claim 2,
wherein said transceiver part is configured to control the ultrasonic probe to perform a second scan which receives the second reflected waves from an area or each of areas whose normal direction is an azimuth direction or an elevation direction.

7. An ultrasonic diagnostic apparatus of claim 1,
wherein said transceiver part is configured to control the ultrasonic probe to perform a second scan which receives the second reflected waves multiple times at timings at which a three dimensional area is divided into temporally equal durations, the three dimensional area being a receiving target of the first reflected waves.

8. An ultrasonic diagnostic apparatus of claim 2,
wherein said transceiver part is configured to control the ultrasonic probe to perform a second scan which receives the second reflected waves multiple times at timings at which a three dimensional area is divided into temporally equal durations, the three dimensional area being a receiving target of the first reflected waves.

9. An ultrasonic diagnostic apparatus of claim 1,
wherein said transceiver part is configured to control the ultrasonic probe to perform a second scan which receives the second reflected waves from a two dimensional area which divides a three dimensional area into two parts and whose normal direction is an azimuth direction or an elevation direction, the three dimensional area being a receiving target of the first reflected waves.

10. An ultrasonic diagnostic apparatus of claim 2,
wherein said transceiver part is configured to control the ultrasonic probe to perform a second scan which receives the second reflected waves from a two dimensional area which divides a three dimensional area into two parts and whose normal direction is an azimuth direction or an elevation direction, the three dimensional area being a receiving target of the first reflected waves.

11. An ultrasonic diagnostic apparatus of claim 1,
wherein said transceiver part is configured to set a receiving condition of the second reflected waves as a frame rate of the two dimensional ultrasonic image data.

12. An ultrasonic diagnostic apparatus of claim 2,
wherein said transceiver part is configured to set a receiving condition of the second reflected waves as a frame rate of the two dimensional ultrasonic image data.

13. An ultrasonic diagnostic apparatus of claim 1,
wherein said transceiver part is configured to be able to adjust a frame rate of the two dimensional ultrasonic image data, according to direction information input from an input device.

14. An ultrasonic diagnostic apparatus of claim 2,
wherein said transceiver part is configured to be able to adjust a frame rate of the two dimensional ultrasonic image data, according to direction information input from an input device.

15. An ultrasonic diagnostic apparatus of claim 1,
wherein said transceiver part is configured to transmit an ultrasonic wave, having a frequency characteristic appropriate for generating ultrasonic contrast volume image data, as each of the first ultrasonic wave and the ultrasonic wave obtained by modulating the amplitude of the first ultrasonic wave, from the ultrasonic probe, in the first scan, and to transmit an ultrasonic wave, having a frequency characteristic appropriate for generating ultrasonic morphological image data, as the second ultrasonic wave, from the ultrasonic probe, in the second scan.

16. An ultrasonic diagnostic apparatus of claim 2,
wherein said transceiver part is configured to transmit an ultrasonic wave, having a frequency characteristic appropriate for generating ultrasonic contrast volume image data, as each of the first ultrasonic wave and the ultrasonic wave obtained by modulating the phase of the first ultrasonic wave, from the ultrasonic probe, in the first scan, and to transmit an ultrasonic wave, having a frequency characteristic appropriate for generating ultrasonic morphological image data, as the second ultrasonic wave, from the ultrasonic probe, in the second scan.

17. An ultrasonic diagnostic apparatus of claim 1,
wherein said transceiver part is configured to control the ultrasonic probe to perform at least one of the first scan and the second scan by a parallel simultaneous receiving method, which simultaneously receives ultrasonic waves from different directions using ultrasonic transducers.

18. An ultrasonic diagnostic apparatus of claim 2,
wherein said transceiver part is configured to control the ultrasonic probe to perform at least one of the first scan and the second scan by a parallel simultaneous receiving method, which simultaneously receives ultrasonic waves from different directions using ultrasonic transducers.

19. An ultrasonic diagnostic method comprising:
controlling an ultrasonic probe to perform:
a first scan which transmits a first ultrasonic wave and an ultrasonic wave, obtained by modulating an amplitude of the first ultrasonic wave, at least one time to each of scanning lines distributed three dimensionally, and receives first reflected waves due to transmissions of the first ultrasonic wave and the ultrasonic wave obtained by modulating the amplitude of the first ultrasonic wave; and
a second scan which transmits a second ultrasonic wave at least one time to each of scanning lines distributed two dimensionally during said first scan, and receives second reflective waves due to transmissions of the second ultrasonic wave;
obtaining first reception signals based on the first reflected waves and second reception signals based on the second reflected waves, from the ultrasonic probe;
generating composite signals by combining the first reception signals; and
generating three dimensional ultrasonic image data based on the composite signals and generating two dimensional ultrasonic image data based on the second reception signals.

20. An ultrasonic diagnostic method comprising:
controlling an ultrasonic probe to perform:
a first scan which transmits a first ultrasonic wave and an ultrasonic wave, obtained by modulating a phase of the first ultrasonic wave, at least one time to each of scanning lines distributed three dimensionally, and receives first reflected waves due to transmissions of the first ultrasonic wave and the ultrasonic wave obtained by modulating the phase of the first ultrasonic wave; and
a second scan which transmits a second ultrasonic wave at least one time to each of scanning lines distributed two dimensionally during said first scan, and receives second reflective waves due to transmissions of the second ultrasonic wave;
obtaining first reception signals based on the first reflected waves and second reception signals based on the second reflected waves, from the ultrasonic probe;
generating composite signals by combining the first reception signals; and
generating three dimensional ultrasonic image data based on the composite signals and generating two dimensional ultrasonic image data based on the second reception signals.
